(19) United States
(12) Patent Application Publication
(10) Pub. No.: US 2022/0036980 A1
(43) Pub. Date: Feb. 3, 2022

(54) METHODS AND APPARATUS TO MONITOR LAB EQUIPMENT

(71) Applicant: Illinois Tool Works Inc., Glenview, IL (US)

(72) Inventors: Sarah M. Beranek, Glenview, IL (US); Benjamin Mangrich, Evanston, IL (US); Julien Noel, Chicago, IL (US)

(21) Appl. No.: 17/387,519

(22) Filed: Jul. 28, 2021

Related U.S. Application Data
(60) Provisional application No. 63/059,610, filed on Jul. 31, 2020.

(51) Int. Cl. G16H 10/40; G16H 40/63; G16H 40/67; G06F 21/73

(52) U.S. Cl. CPC G16H 10/40; G06F 21/73; G16H 40/67; G16H 40/63

(57) ABSTRACT

An example system to monitor lab equipment includes: lab devices configured to perform sectioning, grinding, mounting, polishing, imaging, and/or hardness testing, of specimens; and an equipment monitoring system configured to: receive the data from the lab devices, the data comprising identifiers of the lab devices, operating statuses of the lab devices, current operating cycle information for the lab devices, error codes, and parameters of the operating cycles performed by the lab devices, wherein the lab devices are configured to transmit the data to the equipment monitoring system via a network; based on the error codes, output operator-readable information associated with the lab devices from which the error codes were received; and in response to at least one of the operating statuses, the current operating cycle information, the error codes, or the parameters satisfying an event definition, output an operator-readable notification identifying an operator action item based on the event definition.

### RELATED APPLICATIONS

The present application claims the benefit of U.S. Provisional Patent Application Ser. No. 63/059,610, filed Jul. 31, 2020, entitled “METHODS AND APPARATUS TO MONITOR LAB EQUIPMENT.” The entirety of U.S. Patent Application Ser. No. 63/059,610 is expressly incorporated herein by reference.

### BACKGROUND

The present disclosure relates to monitoring systems and, more particularly, to methods and apparatus to monitor lab equipment.

Limitations and disadvantages of conventional approaches will become apparent to one of skill in the art, through comparison of such approaches with some aspects of the present method and system set forth in the remainder of this disclosure with reference to the drawings.

### SUMMARY

Methods and apparatus to monitor lab equipment are disclosed, substantially as illustrated by and described in connection with at least one of the figures, and as set forth more completely in the claims.

### BRIEF DESCRIPTION OF THE DRAWINGS

These and/or other aspects will become apparent and more readily appreciated from the following description of the exemplary embodiments, taken in conjunction with the accompanying drawings.

FIG. 1 illustrates a block diagram of an example system to monitor lab equipment including example lab devices and an equipment monitoring system, in accordance with aspects of this disclosure.

FIGS. 2A-2D illustrate example user interface screens that may be output by the example equipment monitoring system and/or presented via the example external computing device of FIG. 1 to display equipment monitoring information based on the data provided by the example lab devices.

FIG. 3 is a block diagram of an example computing system that may be used to implement the equipment monitoring system and/or the example external computing system of FIG. 1.

FIG. 4 is a flowchart representative of example machine readable instructions which may be executed by the example equipment monitoring system of FIG. 1 and/or the example computing system of FIG. 3 to monitor lab equipment.

The figures are not necessarily to scale. Where appropriate, similar or identical reference numbers are used to refer to similar or identical components.

### DETAILED DESCRIPTION

Disclosed systems and methods to monitor lab equipment substantially improve the operation and efficiency of material testing laboratory. For example, disclosed systems and methods collect data from multiple laboratory devices at one or more centralized locations, process the data according to event definitions, and output notifications in response to satisfying or triggering the conditions in the event definition. In some disclosed systems and methods, an equipment monitoring system receives error codes, translates and/or interprets the error codes, and presents the translated error codes in human-readable form. In some examples, the equipment monitoring system interprets the error codes using other contextual information to present the human-readable information.

As used herein, the terms “human-readable” and “operator-readable” refer to narrative or descriptive information that is displayed or otherwise presented to a person. Human-readable and operator-readable is distinguished from arbitrarily encoded information, such as an error code that is assigned a label using alphanumeric characters. While the error codes assigned to encoded error information can be observed by a person, the encoded error information is not considered “human-readable” or “operator-readable” as used herein, because the error codes require interpretation (either via training or reference material) to understand the encoded error information.

Disclosed example systems to monitor lab equipment include a plurality of lab devices configured to perform at least one of sectioning, grinding, mounting, polishing, imaging, or hardness testing, of specimens, and an equipment monitoring system. The equipment monitoring system is configured to: receive the data from the lab devices, the data including identifiers of the lab devices, operating statuses of the lab devices, current operating cycle information for the lab devices, error codes, and parameters of the operating cycles performed by the lab devices, in which the lab devices are configured to transmit the data to the equipment monitoring system via a network; based on the error codes, output operator-readable information associated with the lab devices from which the error codes were received; and in response to at least one of the operating statuses, the current operating cycle information, the error codes, or the parameters satisfying an event definition, output an operator-readable notification identifying an operator action item based on the event definition.

In some example systems, the network includes at least one of a local area network, the Internet, or a virtual private network. In some examples, the identifiers include at least one of an equipment name, a serial number, a network address, or a media control access (MAC) address, the operating statuses include at least one of a ready state, an error state, a paused state, or an operating state, the current operating cycle information includes at least one of a number of operating cycles performed by a lab device, an operating cycle start time, an operating cycle end time, or a remaining time in a current operating cycle, and/or the parameters include at least one of a consumable material feed rate, a rotation speed, an applied force, a pressure, a temperature, a process or recipe name, or a consumable supply.

In some example systems, the equipment monitoring system is configured to display a dashboard interface showing representations of a plurality of the lab devices simultaneously. In some example systems, the equipment monitoring system is configured to display at least one of the data from the lab devices or the operation action item via the dashboard interface. In some example systems, the equipment monitoring system is configured to output the notification via the dashboard interface. In some example systems, the equipment monitoring system is configured to generate and display a trend metric based on a selected one or more of the lab devices.

Disclosed example methods to monitor lab equipment involve: receiving, at an equipment monitoring system, data from a plurality of lab devices configured to perform at least one of sectioning, grinding, mounting, polishing, imaging, or hardness testing, of specimens, the data comprising identifiers of the lab devices, operating statuses of the lab devices, current operating cycle information for the lab devices, error codes, and parameters of the operating cycles performed by the lab devices, wherein the lab devices are configured to transmit the data to the equipment monitoring system via a network; based on the error codes, outputting, via the equipment monitoring system, operator-readable information associated with the lab devices from which the error codes were received; and in response to at least one of the operating statuses, the current operating cycle information, the error codes, or the parameters satisfying an event definition, outputting an operator-readable notification identifying an operator action item based on the event definition.

Some example methods further involve displaying a dashboard interface showing representations of a plurality of the lab devices simultaneously. Some example methods further involve displaying at least one of the data from the lab devices or the operation action item via the dashboard interface. Some example methods further involve outputting the notification via the dashboard interface. Some example methods further involve generating and displaying a trend metric based on a selected one or more of the lab devices.

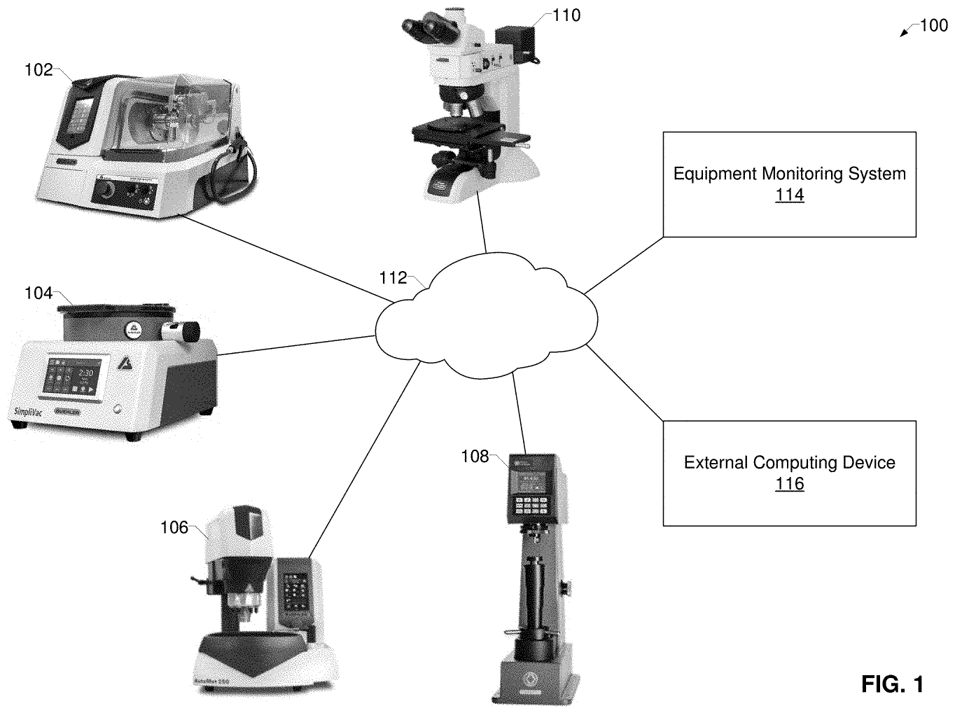

FIG. 1 illustrates a block diagram of an example system 100 to monitor lab equipment. The example system 100 includes multiple lab devices, such as one or more sectioning devices 102, one or more mounting devices 104, one or more grinder/polishers 106, one or more hardness testers 108, and/or one or more imagers 110.

The lab devices 102-110 may be located in a same room or lab, in different rooms or labs in the same building, and/or in different buildings. In particular, the lab devices 102-110 may be connected to a same network 112 via wired and/or wireless connections. The network 112 may include one or more local area networks (LANs). For example, multiple local area networks may be connected via the Internet, such as by using a virtual private network (VPN) or other tunneling to securely couple different LANs.

The example system 100 further includes an equipment monitoring system 114 and an external computing device 116. The equipment monitoring system 114 receives data from the lab devices 102-110. In some examples, the lab devices 102-110 may transmit the data via the network 112. Additionally or alternatively, the lab devices 102-110 may communicate directly with the equipment monitoring system 114. Example data that may be collected from the lab devices 102-110 by equipment monitoring system 114 include identifiers (e.g., names, serial numbers, network addresses, media access control (MAC) addresses, etc.) of the lab devices 102-110, operating statuses (e.g., ready, error, paused, operating, etc.) of the lab devices 102-110, current operating cycle information (e.g., start time, stop time, remaining time in cycle) for the lab devices, error codes, time stamps, device connection status (e.g., connected to the equipment monitoring system 114, disconnected from the equipment monitoring system 114), and/or parameters of the operating cycles performed by the lab devices 102-110.

Operating cycle information may include a number of operating cycles performed by the lab devices 102-110 and/or a state of a current operating cycle. The definition or status of an operating cycle may vary based on the type of lab device 102-110. For example, some equipment may perform testing, material preparation, inspection, and/or other functions.

Example parameters for lab equipment may include consumable material feed rates, rotation speeds, applied force, pressure, temperature, process or recipe name, consumable supply, and/or any other parameters.

The example lab devices 102-110 send the data substantially in real-time and/or as batch data at frequent intervals. Example intervals include any interval up to 24 hours, based on network connectivity for the transmitting lab device 102-110.

The error codes encode status or error information as specified by the lab devices 102-110. Error codes may have specific meanings, or may require additional contextual information to identify the meaning of the error code. Based on the error codes, the example equipment monitoring system 114 outputs operator-readable information associated with the lab devices 102-110 from which the error codes were received. For example, the equipment monitoring system 114 may store the error codes, translate the error codes into human-readable information, and output the human-readable information.

The equipment monitoring system 114 further outputs an operator-readable notification, which identifies an operator action item, based on received operating statuses, current operating cycle information, error codes, received parameters, and/or any other received data. For example, if the received operating statuses, current operating cycle information, error codes, received parameters, and/or any other received data satisfy an event definition, the equipment monitoring system 114 outputs an indication of the operator action item(s) and/or operator-readable notification(s) specified by the event definition. Example operator action item(s) that may be specified by the event definitions may involve reviewing data (e.g., logs, messages, and/or any other information), interacting with the lab device(s) associated with the notification, performing maintenance or service procedures on the lab device(s), and/or any other action items.

In some examples, the equipment monitoring system 114 may output the notification(s) to a local display that is integrated with and/or connected to the equipment monitoring system 114. Additionally or alternatively, the equipment monitoring system 114 may output the notifications to one or more external computing device(s) 116. Example external computing device(s) 116 may include a personal computer, a server, a smartphone, a laptop computer, a workstation, a tablet computer, and/or any other type of computing device. In some examples, the external computing device(s) 116 are communicatively connected to the equipment monitoring system 114 via the network 112, which may include one or more LANs, wireless local area networks (WLANs), wide area networks (WANs), and/or any other type(s) of networks.

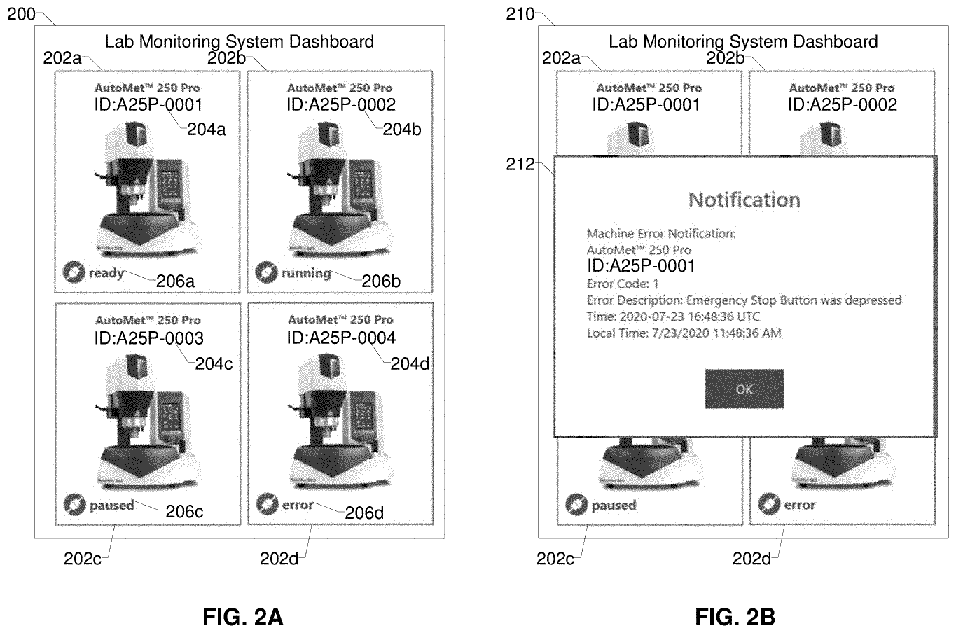

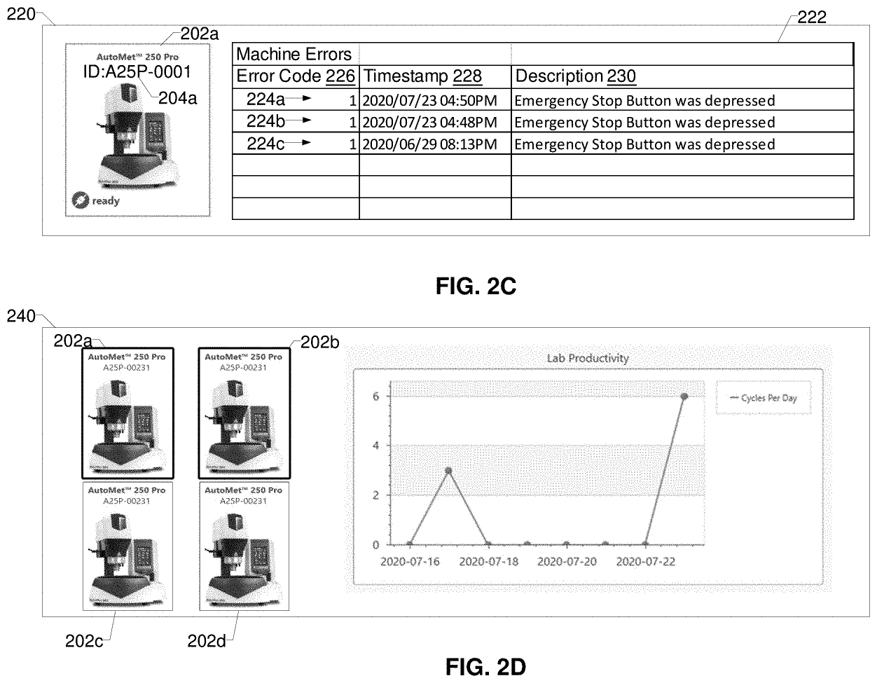

FIG. 2A illustrates an example user interface screen 200 that may be output by the example equipment monitoring system 114 and/or presented via the example external computing device 116 of FIG. 1 to display equipment monitoring information based on the data provided by the example lab devices 102-110. The example user interface screen 200 of FIG. 2A illustrates multiple lab devices 202a-202d.

The screen 200 of FIG. 2A further includes identifiers 204a-204d and statuses 206a-206d corresponding to the respective lab devices 202a-202d. The identifiers 204a-204d may include serial numbers of the lab devices 202a-202d, images of the lab devices 202a-202d, model numbers of the lab devices 202a-202d, customized names of the lab devices 202a-202d, locations of the lab devices 202a-202d, and/or any other identifying information.

The example screen 200 may permit the operator or viewer of the screen 200 to select an icon, link, or other representation of the lab devices 202a-202d to navigate to a different screen or portion of the user interface. For example, selecting the lab device 202a-202d may enable an operator to view additional information about the lab device 202a based on the data received from the lab device 202a.

FIG. 2B illustrates an example user interface screen 210 that may be output by the example equipment monitoring system 114 and/or presented via the example external computing device 116 of FIG. 1 to present a notification 212 in response to an error code transmitted by one of the lab devices 202a. In response to receiving the error code in associated with the lab device 202a, the example equipment monitoring system 114 determines whether the error code, alone or in combination with the identifying data of the lab device 202a, satisfies an event definition. In the illustrated example, an event definition stored by the equipment monitoring system 114 specifies that the notification 212 is to be output (e.g., displayed) in response to receiving the error code. The notification 212 includes an operator-readable translation of the received error code, the lab device 202a associated with the error code, and/or any other information specified by the event definition.

FIG. 2C illustrates an example user interface screen 220 that may be output by the example equipment monitoring system 114 and/or presented via the example external computing device 116 of FIG. 1 to present a log of identified and translated error codes associated with a first one of the lab devices 202a of FIG. 2A. The example screen 220 includes the identifier 204a of the lab device 202a and a log 222 of machine errors received from the lab device 202a. The example log 222 includes three entries 224a, 224b, 224c corresponding to the receipt of an error code from the lab device 202a. Each of the example entries 224a-224c specifies the error code 226, a timestamp 228, and a human-readable translation 230 (e.g., an interpretation or description of the error code 226).

FIG. 2D illustrates an example user interface screen 240 that may be output by the example equipment monitoring system 114 and/or presented via the example external computing device 116 of FIG. 1 to display a trend graph 242 of data received from one or more of the lab devices 202a-202d. The example trend graph 242 provides a customizable view of the data received from the lab devices 202a-202d over a time period.

The example trend graph 242 illustrates a lab productivity metric 244 on a per-day basis for the lab devices 202a-202b, which may be determined based on cycle data transmitted by the lab devices 202a-202b. The equipment monitoring system 114 may permit the operator of the interface to specify the time period, the data displayed, the lab devices 202a-202d from which the data is to be considered, and/or any other aspects of the trend graph 242. For example, the operator may select any one or more of the lab devices 202a-202d for inclusion in the data used to generate the trend graph 242.

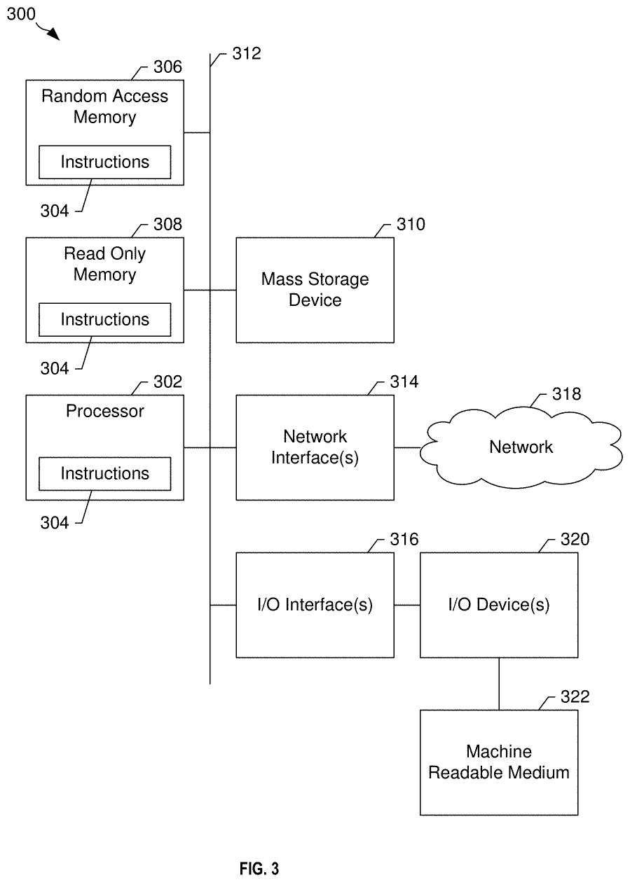

FIG. 3 is a block diagram of an example computing system 300 that may be used to implement the equipment monitoring system 114 and/or the example external computing system 116 of FIG. 1. The example computing system 300 may be implemented using a personal computer, a server, a smartphone, a laptop computer, a workstation, a tablet computer, and/or any other type of computing device.

The example computing system 300 of FIG. 3 includes a processor 302. The example processor 302 may be any general purpose central processing unit (CPU) from any manufacturer. In some other examples, the processor 302 may include one or more specialized processing units, such as RISC processors with an ARM core, graphic processing units, digital signal processors, and/or system-on-chips (SoC). The processor 302 executes machine readable instructions 304 that may be stored locally at the processor (e.g., in an included cache or SoC), in a random access memory 306 (or other volatile memory), in a read only memory 308 (or other non-volatile memory such as FLASH memory), and/or in a mass storage device 310. The example mass storage device 310 may be a hard drive, a solid state storage drive, a hybrid drive, a RAID array, and/or any other mass data storage device.

A bus 312 enables communications between the processor 302, the RAM 306, the ROM 308, the mass storage device 310, a network interface 314, and/or an input/output interface 316.

The example network interface 314 includes hardware, firmware, and/or software to connect the computing system 300 to a communications network 318 such as the Internet. For example, the network interface 314 may include IEEE 302.X-compliant wireless and/or wired communications hardware for transmitting and/or receiving communications.

The example I/O interface 316 of FIG. 3 includes hardware, firmware, and/or software to connect one or more input/output devices 320 to the processor 302 for providing input to the processor 302 and/or providing output from the processor 302. For example, the I/O interface 316 may include a graphics processing unit for interfacing with a display device, a universal serial bus port for interfacing with one or more USB-compliant devices, a FireWire, a field bus, and/or any other type of interface. Example I/O device(s) 320 may include a keyboard, a keypad, a mouse, a trackball, a pointing device, a microphone, an audio speaker, an optical media drive, a multi-touch touch screen, a gesture recognition interface, a display device, a magnetic media drive, and/or any other type of input and/or output device.

The example computing system 300 may access a non-transitory machine readable medium 322 via the I/O interface 316 and/or the I/O device(s) 320. Examples of the machine readable medium 322 of FIG. 3 include optical discs (e.g., compact discs (CDs), digital versatile/video discs (DVDs), Blu-ray discs, etc.), magnetic media (e.g., floppy disks), portable storage media (e.g., portable flash drives, secure digital (SD) cards, etc.), and/or any other type of removable and/or installed machine readable media.

Example wireless interfaces, protocols, and/or standards that may be supported and/or used by the network interface(s) 314 and/or the I/O interface(s) 316, include wireless personal area network (WPAN) protocols, such as Bluetooth (IEEE 302.15); near field communication (NFC) standards; wireless local area network (WLAN) protocols, such as WiFi (IEEE 302.11); cellular standards, such as 2G/2G+ (e.g., GSM/GPRS/EDGE, and IS-95 or cdmaOne) and/or 2G/2G+ (e.g., CDMA2000, UMTS, and HSPA); 4G standards, such as WiMAX (IEEE 302.16) and LTE; Ultra-Wideband (UWB); etc. Example wired interfaces, protocols, and/or standards that may be supported and/or used by the network interface(s) 314 and/or the I/O interface(s) 316, such as to communicate with the display device(s), include comprise Ethernet (IEEE 302.3), Fiber Distributed Data Interface (FDDI), Integrated Services Digital Network (ISDN), cable television and/or internet (ATSC, DVB-C, DOCSIS), Universal Serial Bus (USB) based interfaces, etc.

The processor 302, the network interface(s) 314, and/or the I/O interface(s) 316 may perform signal processing operations such as, for example, filtering, amplification, analog-to-digital conversion and/or digital-to-analog conversion, up-conversion/down-conversion of baseband signals, encoding/decoding, encryption/decryption, modulation/demodulation, and/or any other appropriate signal processing.

The computing system 300 may use one or more antennas for wireless communications and/or one or more wired port(s) for wired communications. The antenna(s) may be any type of antenna (e.g., directional antennas, omnidirectional antennas, multi-input multi-output (MIMO) antennas, etc.) suited for the frequencies, power levels, diversity, and/or other parameters required for the wireless interfaces and/or protocols used to communicate. The port(s) may include any type of connectors suited for the communications over wired interfaces/protocols supported by the computing system 300. For example, the port(s) may include an Ethernet over twisted pair port, a USB port, an HDMI port, a passive optical network (PON) port, and/or any other suitable port for interfacing with a wired or optical cable.

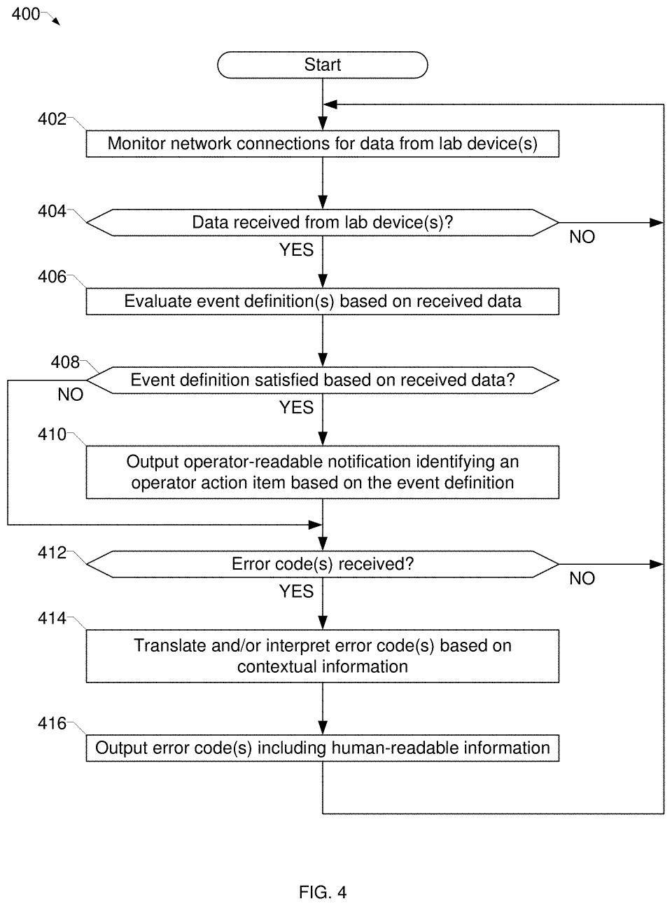

FIG. 4 is a flowchart representative of example machine readable instructions 400 which may be executed by the example equipment monitoring system 114 of FIG. 1 and/or the example computing system 300 of FIG. 3 to monitor lab equipment. The example instructions 400 may be implemented as the instructions 304 and performed by the example processor 302 of FIG. 3.

At block 402, the equipment monitoring system 114 (e.g., via the processor 302) monitors connections for data from one or more lab device(s). For example, the lab device(s) 102-110 of FIG. 1 may be configured to transmit (e.g., periodically, aperiodically, in response to an event, etc.) data about the lab device(s) 102-110 to the equipment monitoring system 114 directly and/or via the network 112. The data may include identifiers of the lab devices 102-110, operating statuses of the lab devices 102-110, current operating cycle information for the lab devices 102-110, error codes, and parameters of the operating cycles performed by the lab devices 102-110.

At block 404, the equipment monitoring system 114 determines whether data has been received from any lab device(s) 102-110. If data has not been received (block 404), control returns to block 402 to continue monitoring.

When data is received (block 404), at block 406 the equipment monitoring system 114 evaluates event definitions based on the received data. For example, the equipment monitoring system 114 may determine whether any event definitions stored by the equipment monitoring system 114 are satisfied by the received data, either alone or in combination with previously received data. The event definitions may be predetermined based on the types of lab devices 102-110 connected to the equipment monitoring system 114, and/or customized by an operator, supervisor, or other personnel.

At block 408, the equipment monitoring system 114 determines whether any event definitions are satisfied based on the received data. If an event definition is satisfied (block 408), at block 410 the equipment monitoring system 114 outputs an operator-readable notification identifying an operator action item based on the event definition. For example, the equipment monitoring system 114 may output a notification 212 as shown in FIG. 2B. The notification may be output via a local display, transmitted to the lab device 102-110 for display at the lab device 102-110, and/or transmitted to one or more external computing devices 116.

If multiple event definitions are satisfied (block 408), multiple operator-readable notifications may be output.

After outputting the operator-readable notification (block 410), or if no event definitions are satisfied (block 408), at block 412 the equipment monitoring system 114 determines whether any error codes are received in the data. If any error codes are received (block 412), at block 414 the equipment monitoring system 114 translates the error code(s) and/or interprets the error code(s) based on contextual information. For example, the error codes may be translated where contextual information is not needed (e.g., an emergency stop button is pressed). The error codes may be interpreted based on additional contextual information, such as determining a consumable material supply level to interpret an error code identifying a problem during an operating cycle.

At block 416, the equipment monitoring system 114 outputs the error code(s) including human-readable information. Control then returns to block 402 to continue monitoring.

The present methods and systems may be realized in hardware, software, and/or a combination of hardware and software. The present methods and/or systems may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may include a general-purpose computing system with a program or other code that, when being loaded and executed, controls the computing system such that it carries out the methods described herein. Another typical implementation may comprise an application specific integrated circuit or chip. Some implementations may comprise a non-transitory machine-readable (e.g., computer readable) medium (e.g., FLASH drive, optical disk, magnetic storage disk, or the like) having stored thereon one or more lines of code executable by a machine, thereby causing the machine to perform processes as described herein. As used herein, the term “non-transitory machine-readable medium” is defined to include all types of machine readable storage media and to exclude propagating signals.

As utilized herein the terms “circuits” and “circuitry” refer to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled or not enabled (e.g., by a user-configurable setting, factory trim, etc.).

While the present method and/or system has been described with reference to certain implementations, it will be understood by those skilled in the art that various changes may be made and equivalents may be substituted without departing from the scope of the present method and/or system. For example, block and/or components of disclosed examples may be combined, divided, re-arranged, and/or otherwise modified. In addition, many modifications may be made to adapt a particular situation or material to the teachings of the present disclosure without departing from its scope. Therefore, the present method and/or system are not limited to the particular implementations disclosed. Instead, the present method and/or system will include all implementations falling within the scope of the appended claims, both literally and under the doctrine of equivalents.

What is claimed is:

1. A system to monitor lab equipment, the system comprising:
a plurality of lab devices configured to perform at least one of sectioning, grinding, mounting, polishing, imaging, or hardness testing, of specimens; and
an equipment monitoring system configured to:
receive the data from the lab devices, the data comprising identifiers of the lab devices, operating statuses of the lab devices, current operating cycle information for the lab devices, error codes, and parameters of the operating cycles performed by the lab devices, wherein the lab devices are configured to transmit the data to the equipment monitoring system via a network;
based on the error codes, output operator-readable information associated with the lab devices from which the error codes were received; and
in response to at least one of the operating statuses, the current operating cycle information, the error codes, or the parameters satisfying an event definition, output an operator-readable notification identifying an operator action item based on the event definition.

2. The system as defined in claim 1, wherein the network comprises at least one of a local area network, the Internet, or a virtual private network.

3. The system as defined in claim 1, wherein the identifiers comprise at least one of an equipment name, a serial number, a network address, or a media control access (MAC) address.

4. The system as defined in claim 1, wherein the operating statuses comprise at least one of a ready state, an error state, a paused state, or an operating state.

5. The system as defined in claim 1, wherein the current operating cycle information comprises at least one of a number of operating cycles performed by a lab device, an operating cycle start time, an operating cycle end time, or a remaining time in a current operating cycle.

6. The system as defined in claim 1, wherein the parameters comprise at least one of a consumable material feed rate, a rotation speed, an applied force, a pressure, a temperature, a process or recipe name, or a consumable supply.

7. The system as defined in claim 1, wherein the equipment monitoring system is configured to display a dashboard interface showing representations of a plurality of the lab devices simultaneously.

8. The system as defined in claim 7, wherein the equipment monitoring system is configured to display at least one of the data from the lab devices or the operation action item via the dashboard interface.

9. The system as defined in claim 7, wherein the equipment monitoring system is configured to output the notification via the dashboard interface.

10. The system as defined in claim 1, wherein the equipment monitoring system is configured to generate and display a trend metric based on a selected one or more of the lab devices.

11. A method to monitor lab equipment, the method comprising:
receiving, at an equipment monitoring system, data from a plurality of lab devices configured to perform at least one of sectioning, grinding, mounting, polishing, imaging, or hardness testing, of specimens, the data comprising identifiers of the lab devices, operating statuses of the lab devices, current operating cycle information for the lab devices, error codes, and parameters of the operating cycles performed by the lab devices, wherein the lab devices are configured to transmit the data to the equipment monitoring system via a network;
based on the error codes, outputting, via the equipment monitoring system, operator-readable information associated with the lab devices from which the error codes were received; and
in response to at least one of the operating statuses, the current operating cycle information, the error codes, or the parameters satisfying an event definition, outputting an operator-readable notification identifying an operator action item based on the event definition.

12. The method as defined in claim 11, further comprising displaying a dashboard interface showing representations of a plurality of the lab devices simultaneously.

13. The method as defined in claim 12, further comprising displaying at least one of the data from the lab devices or the operation action item via the dashboard interface.

14. The method as defined in claim 12, further comprising outputting the notification via the dashboard interface.

15. The method as defined in claim 11, further comprising generating and displaying a trend metric based on a selected one or more of the lab devices.
